(19) United States
(12) Patent Application Publication
(10) Pub. No.: US 2006/0165621 A1
(43) Pub. Date: Jul. 27, 2006

(54) cosmetic composition comprising fluorescent nanoparticles as pigments

(76) Inventors: Louis Dubertret, Paris (FR); Benoit Dubertret, Paris (FR)

Correspondence Address: STITES & HARBISON PLLC, 1199 NORTH FAIRFAX STREET, SUITE 900, ALEXANDRIA, VA, 22314, US

(21) Appl. No.: 10/536,736

(22) PCT Filed: Nov. 28, 2003
(86) PCT No.: PCT/FR03/03525

§ 371 Date: May 27, 2005

(30) Foreign Application Priority Data
Nov. 28, 2002 (FR) 0214987

(51) Int. Cl. A61K 8/19

(52) U.S. Cl. 424/63; 977/926

(57) ABSTRACT

The invention concerns a make-up composition comprising as pigment cosmetically acceptable fluorescent semi-conductive nanoparticles in a cosmetic support.

The present invention concerns a novel cosmetic composition, in particular for make-up, comprising as a pigment fluorescent nanoparticles consisting of a semiconductor, called “quantum dots”. It also concerns a method for producing such a composition.

It is commercially desirable to provide cosmetic products having unique decorative, functional and aesthetic effects. These effects are generally obtained by using pigments, glasses or other products providing iridescent, luminescent or reflective effects when they are mixed with cosmetic products.

Make-up compositions in particular, such as in particular mascaras, blushes, eye shadow, lipsticks, nail varnishes or lacquers, consist of a suitable cosmetic vehicle and different colouring agents designed to provide the compositions with a certain colour before and/or after they are applied to the skin, lips and/or skin appendages.

In order to create colours, a quite limited range of colouring agents is used at the present time, in particular pigments such as lakes, inorganic pigments or nacreous pigments. Lakes enable vivid colours to be obtained. However, most of these lakes have poor resistance to light, temperature and/or pH. Some also have the disadvantage of staining the skin in an unsightly manner after application, by colorant discharge. Inorganic pigments, in particular inorganic oxides, are very stable but give colours that are rather dull and pale. Nacreous pigments make it possible to obtain various colours, but ones that are never intense, with effects that are iridescent but are often quite weak, and above all the colour effect is mainly visible at a certain given angle corresponding to specular reflection.

It has been discovered that semiconductor nanocrystals exhibit quantum effects that result in special luminescent properties. In point of fact, these “quantum dots” emit, by fluorescence, when they are excited by visible or ultraviolet light, light of which the wavelength and therefore the colour is a function of their size.

At the present time, the use of these fluorescent nanoparticles has been envisaged for labelling biomolecules, in particular in the field of molecular biology.

However, the development of these applications has encountered the difficulty of making fluorescent nanoparticles compatible with the cosmetic vehicle and in particular of ensuring homogeneous and stable distribution while preserving other properties such as colloidal stability, low toxicity and quantum yield. This presents a particular problem concerning vehicles having an aqueous medium.

In point of fact, the method according to U.S. Pat. No. 6,319,426, enabling fluorescent nanoparticles to be obtained having a narrow particle size distribution, comprises covering the nanoparticle with a hydrophobic ligand. These fluorescent nanoparticles thus have a low affinity for water and are therefore difficult to incorporate in hydrophilic media.

In order to make fluorescent nanoparticles compatible with aqueous media, it has been proposed to exchange the hydrophobic ligands surrounding the fluorescent nanoparticles with a ligand monolayer carrying at one end a hydrophilic group and at the other end a thiol group that forms a bond to the surface of the quantum dot (Chan et al. Science (1998), 281: 2016, U.S. Pat. No. 6,319,426). However, the fluorescent nanoparticles obtained in this way have insufficient stability.

It has also been proposed to encapsulate the fluorescent nanoparticles in a silica shell that is modified at the surface so as to give rise to silane groups (M. Bruchez, et al. Science (1998), 281: 2013). However, this method has the disadvantage of being long and difficult.

The use of micelles for solubilising fluorescent nanoparticles in water is described in U.S. Pat. No. 6,319,426. It is proposed therein to form micelles by using sodium dioctylsulphosuccinate or Brij. However, these micelles prove to be unstable in aqueous solution.

One object of the present invention is thus to provide a hydrophilic cosmetic composition that comprises, as the pigment, fluorescent nanoparticles that overcome the disadvantages mentioned. The invention also relates to a method for producing such cosmetic compositions. The compositions according to the invention have a certain number of valuable characteristics.

On the one hand, they have a colour that comes not from a phenomenon of the absorption of ambient light, but an emission of light by the fluorescent nanoparticles. This emission provides a more vivid and intense colouration.

Since the wavelength of the light emitted by these particles is a function of the size of the particles, this can be easily varied over all the spectrum. It is therefore possible to obtain different colours with particles of identical chemical nature. Accordingly, problems of compatibility between the base cosmetic composition and the different pigments is overcome.

It is of course possible to prepare compositions comprising fluorescent nanoparticles with different sizes and/or having a wide particle size distribution so as to provide composite colour compositions.

Compositions are however generally preferred comprising fluorescent nanoparticles with only one size having a narrow particle size distribution, which provides a clearer and more intense colour.

In the following description, pigments are understood to mean particles that are insoluble in the medium that makes up the cosmetic composition, that is to say dispersed or solid in one of the phases of the said medium and acting as a colouration (creation or modification of colour tints) and/or the opacity of the said composition.

Fluorescent nanoparticles that can be incorporated as a pigment in cosmetic compositions comprise semiconductor compounds that are preferably cosmetically acceptable.

Cosmetically acceptable compounds are understood to be those that are non-toxic for humans when applied to the skin, eyelashes, nails or hair.

These semiconductors comprise cosmetically acceptable compounds of group IV of the periodic system of elements, of groups II-VI and of group III-V. The semiconductor may also comprise mixtures of these semiconductors such as in particular CdSe/CdS, CdTe/ZnS, CdTe/ZnSe or InAs/ZnSe.

Among the semiconductors of groups II-VI, mention may be made in particular of MgS, MgSe, MgSe, MgTe, CaS, CaSe, CaTe, SrS, SrSe, SrTe, BaS, BaSe, BaTe, ZnS, ZnSe, ZnTe, CdS, CdSe, HgS, HgSe and HgTe.

Among the semiconductors of groups III-V, GaAs, GaN, GaP, GaSb, InGaAs, InP, InN, InSb, InAs, AlAs, AlP, AlSb and AlS are preferred.

Finally, among the semiconductors of group IV, Ge, Pb and Si are particularly suitable.

According to one particular embodiment, the nanoparticle comprises a semiconductor encapsulated in one or more other materials. It then has a structure known as a core/shell type (it being possible for the shell to be multilayered). Preferably, but not compulsorily, the shell also comprises one or more semiconductors (as for example in the case of a CdSe core encapsulated in ZnSe and then ZnS; see the article by P. Reiss (Reiss P., Carayon S. et al (2003). “Low polydispersity core/shell nanocrystals of CdSe/ZnSe and CdSe/ZnSe/ZnS type: preparation and optical studies”. Synthetic Metals 139 (3); 649-652).

This type of fluorescent nanoparticles has a particularly high quantum yield at ambient temperature. It exhibits another advantage of preserving the core from physical and chemical interactions, and this contributes to a much higher stability. This aspect is particularly valuable within the context of cosmetic applications, since it makes it possible to choose the core material from all semiconductors, independently of their toxicity. Limitation to cosmetically acceptable semiconductors then applies in this case only to shell materials.

For fluorescent nanoparticles of the core/shell type, the core comprises, as the semiconductor, MgS, MgSe, MgTe, CaS, CaSe, CaTe, SrS, SrSe, SrTe, BaS, BaTe, ZnS, ZnSe, ZnTe, CdS, CdSe, CdTe, HgS, HgSe, HgTe, GaAs, GaN, GaP, GaSb, InGaAs, InP, InN, InSb, InAs, AlAs, AlP, AlSb, AlS, PbS, PbSe, Ge, Si or one of the mixtures thereof.

Preferably, the shell of fluorescent nanoparticles also comprises a semiconductor. It may then consist in particular of ZnO, ZnS, ZnSe, ZnTe, CdO, CdS, CdSe, CdTe, MgS, MgSe, GaS, GaN, GaP, GaAs, GaSb, InAs, InN, InP, InSb, AlAs, AlN, AlP, AlSb, or one of the mixtures thereof.

Encapsulation may be carried out for example by epitaxial growth as described for example in Peng et al., J. Am. Chem. Soc., (1997) 119: 7019-7029.

Generally, fluorescent nanoparticles have a mean size of between 1.5 and 50 nm, preferably between 2 and 40 nm. In the case of encapsulated fluorescent nanoparticles of the core/shell type, the core preferably has a mean size of between 1.5 and 10 nm and the encapsulating layer (shell) a thickness of 1 to 10 monolayers.

The size of these fluorescent nanoparticles prevents any migration through the cutaneous barrier. The size may be controlled during their production, for example by using the methods described in the following patents: U.S. Pat. No 5,751,018, U.S. Pat. No. 5,505,928 and U.S. Pat. No. 5,262,357.

Consequently, the emission spectrum of the fluorescent nanoparticles may be controlled by their particle size distribution, their mean size and their composition and, where appropriate, with the aid of encapsulating layers.

Adjustment of these parameters then makes it possible to obtain a spectrum corresponding to the colouration that it is desired to confer on the cosmetic composition.

According to one particular embodiment of the invention, the fluorescent nanoparticles are encapsulated in a specific micelle so as to make them compatible with a hydrophilic medium.

One or more fluorescent nanoparticles are then encapsulated in a micelle with a size of between 5 and 45 nm, which comprises a hydrophilic envelope having a plurality of hydrophilic parts and a hydrophobic core comprising a plurality of hydrophobic parts, each of the hydrophobic parts having at least one chain with at least 8 carbon atoms, and each of the parts having at least 24 carbon atoms for all the chains.

Also, preferably, one or more fluorescent nanoparticles previously coated with a hydrophobic ligand are then complexed into a micelle with a size of between 5 and 45 nm, the micelle being formed of a hydrophobic core and a hydrophilic envelope, the hydrophobic core containing a plurality of hydrophobic groups, the envelope containing a plurality of hydrophilic groups, each hydrophobic group containing at least one chain, each chain comprising at least 8 carbon atoms, the number of carbon atoms for all the hydrophobic chains of a single hydrophobic group being greater than or equal to 24.

Preferably, the hydrophobic group is formed of two carbon chains. The hydrophilic group is preferably a polysaccharide such as agarose, dextran, starch, cellulose, amylose or amylopectin. It may however also consist of synthetic polymers, such as for example polyethylene glycol and other hydrophilic monomers. The micelle is preferably formed of block copolymers and in particular phospholipid-PEG such as those used in the article published by Dubertret, B., Skourides P., et al (2002). “In vivo imaging of quantum dots encapsulated in phospholipid micelles”. Science 298 (5599): 1759-1762.

By reason of the hydrophobic coating for fluorescent nanoparticles, the hydrophobic groups are then oriented towards the nanoparticle and the hydrophilic groups outwards, thus enabling them to be solubilised in an aqueous solution.

These fluorescent nanoparticles in micelles also exhibit great stability and are biocompatible, i.e. non-toxic, and have a low non-specific adsorption. In other words, they do not aggregate together or with other molecules, or only to a small degree.

Some cosmetic vehicles are hydrophobic (varnishes, lacquers etc) and do not require solubilisation of the fluorescent nanoparticles in aqueous media. In this case, the fluorescent nanoparticles are covered with hydrophobic ligands or hydrophobic polymers so as to prevent them aggregating and so as to protect them against any fillers present in solution.

The compositions according to the invention additionally include fluorescent semiconductor nanoparticles in a cosmetic vehicle.

This cosmetic vehicle may be monophasic. It is however normal in the cosmetics field for the vehicle to have two or even more phases. In any case, the cosmetic vehicle has a continuous hydrophilic or hydrophobic phase.

The quantity of fluorescent nanoparticles introduced into the cosmetic vehicle, as determined by a person skilled in the art, is in particular a function of the destination of the composition; it may extend from 0.01% to 50% by weight, preferably 0.5 to 25% by weight based on the total weight of the composition.

In the case of cosmetic vehicles comprising a hydrophilic phase and a hydrophobic phase, the fluorescent nanoparticles will be concentrated according to their previous treatment, in one or other of the phases. Thus, it is possible to prepare cosmetic compositions containing one and/or both of the different phases of fluorescent nanoparticles, and this makes it possible to obtain certain special visual effects.

The compositions according to the invention may be useful in cosmetic products, such as in particular make-up products, for application to the skin, face or body, or for cosmetic treatments of nails, eyelashes, eyebrows, hair and lips.

According to one preferred embodiment, the cosmetic composition is a make-up composition. Make-up compositions generally include at least one hydrophobic phase. They may however also include a continuous or dispersed hydrophilic phase. These phases may be in the liquid, gaseous and/or solid state.

Such compositions comprise for example nail varnish, lipstick, mascara, foundation creams, rouge, eye-shadow, hair lacquers etc. These compositions also make it possible to obtain very special visual effects while being capable of providing suitable care and protection.

The composition of the invention may be in the form of a product intended to be applied to the skin of the body as well as of the face, to the hair, eyelashes, eyebrows and to the nails. The composition according to the invention thus contains a cosmetically acceptable medium compatible with all the keratin materials with which it comes into contact.

When the composition is in the form of an emulsion, the composition may optionally additionally include a surfactant, preferably in a quantity of 0 to 30% by weight, preferably from 0.01 to 30% by weight based on the total weight of the composition.

The emulsion may be a single or multiple emulsion, in particular a W/O, O/W, W/O/W and O/W/O emulsion. It is understood that the fluorescent nanoparticles may be present in any one or more of these phases.

According to the application envisaged, the composition may also additionally include at least one film-forming polymer, in particular for mascaras, eyeliner or hair compositions of the lacquer type. The polymer may be dissolved or dispersed in a cosmetically acceptable medium and possibly associated with at least one coalescing agent and/or at least one plasticiser.

The composition according to the invention may also include a fat phase that contains in particular at least one liquid fat and/or at least one fat that is solid at ambient temperature and atmospheric pressure.

Liquid fats, often called oils, may constitute 0 to 90%, preferably 0.01 to 85% by weight based on the total weight of the fat phase.

Solid or pasty fats may be chosen in particular from waxes, gums and mixtures thereof.

As an indication, the composition may contain 0 to 50%, preferably 0.01 to 40%, and in particular 0.1 to 30% by weight of solid or pasty fats based on the total weight of the composition.

The composition according to the invention may additionally include 0 to 30%, preferably 0.01 to 35% by weight of other particles based on the total weight of the composition. These particles may in particular be a pigment other than the fluorescent nanoparticles, a pearl pigment or a filler. The presence of these other particles makes it possible in particular to make the composition opaque.

In addition, the composition according to the invention may include ingredients conventionally present in such compositions, such as preservatives, antioxidants, thickeners, perfumes, moisturising agents, sun filters, essential oils, vegetable extracts and vitamins.

According to another feature, the invention provides a method for preparing such a cosmetic composition, comprising steps consisting of:

i) provision of fluorescent nanoparticles;

ii) if necessary, a previous compatibility treatment of the fluorescent nanoparticles; and

iii) introduction of the fluorescent nanoparticles treated in this way into a cosmetic vehicle.

The previous compatibility treatment of the fluorescent nanoparticles is only necessary in as far as they are incompatible with the cosmetic vehicle.

Generally, it is understood that the fluorescent nanoparticles may be previously incorporated in one of the other constituents of the cosmetic composition or then incorporated in the finished cosmetic vehicle.

The invention will be better understood in the light of the following examples given in a non-limiting manner.

### EXAMPLE 1

### Preparation of Fluorescent Nanoparticles

Fluorescent nanoparticles of CdSe/ZnS were obtained according to articles by Murray C. B., Norris D. J. et al (1993) “Synthesis and Characterization of Nearly Monodisperse CdE (E=S, Se, Te) Semiconductor Nanocrystallites”, Journal of the American Chemical Society 115(19); 8706-8715 and Hines, M. A. and GuyotSionnest P. (1996), “Synthesis and characterization of strongly luminescing ZnS-capped CdSe nanocrystals”, Journal of Physical Chemistry 100(2); 468-471.

200 μl of a solution of dimethylcadmium were mixed with 16 ml of tri-n-octylphosphine (TOP) and 4 ml of a 1M solution of Se in TOP. This mixture was rapidly injected, with the exclusion of air, into a heated flask at 350° C. containing 30 g of tri-n-octylphosphine Oxide (TOPO). After injection, the solution was brought to ambient temperature. The fluorescent nanoparticles formed were isolated by precipitation from methanol after addition of 5 ml of butanol and the liquid phase was separated by centrifuging. The fluorescent nanoparticles were then suspended in 15 ml of hexane. Fluorescent nanoparticles were obtained with a mean diameter of 2 nm.

### EXAMPLE 2

### Preparation of Core/shell Fluorescent Nanoparticles

In order to form core/shell fluorescent nanoparticles, 250 μl of the solution obtained above (core) were injected into 10 ml of TOPO. After heating to 140° C., a solution containing 5 ml of TOP, 100 μl of diethylzinc and 100 μl of hexamethyldisilthiane were injected drop-by-drop. After injection, the flask was cooled to 90° C. and kept at this temperature for one hour. The core/shell fluorescent nanoparticles were precipitated with methanol and suspended in a 15 ml hexane solution. Fluorescent nanoparticles were obtained having a mean diameter of approximately 2 nm and emitting at around 520 nm.

### EXAMPLE 3

### Preparation of a Fluorescent Nail Varnish

100 μl of the solution of core/shell fluorescent nanoparticles obtained in example 2 were mixed in a suitable container with 5 μl of TOP. This mixture was added to 1 ml of colourless nail hardener (“nail hardener” from C. Dior) or a pure varnish (“pure long-lasting varnish free from formalin, toluene and rosin” from PHAS hypoallergenic)

After stirring vigorously for a plurality of minutes, a homogeneous coloured mixture was obtained that could be applied to nails in the same way as a conventional varnish. The varnish exhibited fluorescence in the green region under the effect of UV light.

In the same way, varnishes were prepared containing fluorescent nanoparticles of CdSe/ZnS of which the size varied between 1.5 and 6 nm in diameter so as to give varnishes that fluoresced in the blue region (1.5 nm in diameter), the green region (3 nm in diameter), the orange region (5 nm in diameter) or the red region (6 nm in diameter).

The drying, adhesion, brilliance and strength of the varnish were not affected by the presence of the fluorescent nanoparticles.

### EXAMPLE 4

### Preparation of a Skin Cream Containing Fluorescent Nanoparticles

1 ml of Toleriane cream (La Roche-Posay) were mixed in a suitable container with 100 μl of a solution of fluorescent nanoparticles prepared above encapsulated in phospholipid micelles obtained by following the protocol described in the article by Dubertret et al (In vivo imaging of quantum dots encapsulated in phospholipid micelles Science 298, 1759-1762).

After stirring vigorously, a fluorescent cream was obtained of which the emission depended on the fluorescent nanoparticles that were incorporated therein. The fluorescent cream was stable for a plurality of months.

1. Make-up composition comprising, as the pigment, cosmetically acceptable fluorescent semiconductor nanoparticles in a cosmetic vehicle.

2. Composition according to claim 1, wherein the cosmetic vehicle comprises a continuous hydrophobic phase.

3. Composition according to claim 1, wherein the cosmetic vehicle comprises a continuous hydrophilic phase.

4. Composition according to claim 1, wherein the cosmetic vehicle is an emulsion.

5. Composition according to claim 4, wherein the cosmetic vehicle is a W/O, O/W, W/O/W or O/W/O emulsion.

6. Composition according to claim 1, wherein the fluorescent semiconductor nanoparticles are dispersed in the hydrophobic phase of the cosmetic vehicle.

7. Composition according to claim 1, wherein the fluorescent semiconductor nanoparticles are dispersed in the hydrophilic phase of the cosmetic vehicle.

8. Composition according to claim 1, wherein the fluorescent nanoparticles comprise a semiconductor of groups 11-VI chosen from MgS, MgSe, MgSe, MgTe, CaS, CaSe, CaTe, SrS, SrSe, SrTe, BaS, BaSe, BaTe, ZnS, ZnSe, ZnTe, US, CdSe, HgS, HgSe and HgTe.

9. Composition according to claim 1, wherein the fluorescent nanoparticles comprise a semiconductor of groups I11-V chosen from GaAs, GaN, GaP, GaSb, InGaAs, InP, InN, InSb, InAs, AlAs, AlP, AlSb and AlS.

10. Composition according to claim 1, wherein the fluorescent nanoparticles comprise a semiconductor of group VI chosen from Ge, Pb and Si.

11. Composition according to claim 1, wherein the fluorescent nanoparticles comprise a mixture of a plurality of semiconductors.

12. Composition according to claim 11, wherein the semiconductor mixture is chosen from CdSe/CdS, CdTe/ZnS, CdTe/ZnSe or InAs/ZnSe.

13. Composition according to claim 1, wherein the fluorescent nanoparticles have a core/shell structure, it being possible for the shell to be formed of a plurality of layers.

14. Composition according to claim 13, wherein the core of the fluorescent nanoparticles is composed of MgS, MgSe, MgTe, CaS, CaSe, CaTe, SrS, SrSe, SrTe, BaS, BaTe, ZnS, ZnSe, ZnTe, CdS, CdSe, CdTe, HgS, HgSe, HgTe, GaAs, GaN, GaP, GaSb, InGaAs, InP, InN, InSb, InAs, AlAs, AlP, AlSb, AlS, PbS, PbSe, Ge, Si or one of the mixtures thereof.

15. Composition according to claim 13, wherein the shell of the fluorescent nanoparticles is composed of ZnO, ZnS, ZnSe, ZnTe, CdO, CdS, CdSe, CdTe, MgS, MgSe, GaAs, GaN, GaP, GaAs, GaSb, InAs, InN, InP, InSb, AlAs, AlN, AlP, AlSb or one of the mixtures thereof.

16. Composition according to claim 13 wherein the shell has a thickness of between 1 and 10 monolayers.

17. Composition according to claim 1, wherein one or more fluorescent nanoparticles have been previously coated with a hydrophobic ligand and then complexed into a micelle with a size of between 5 and 45 nm, the micelle being formed of a hydrophobic core and a hydrophilic envelope, the hydrophobic core containing a plurality of hydrophobic groups, the envelope containing a plurality of hydrophilic groups, each hydrophobic group containing at least one chain, each chain comprising at least 8 carbon atoms, the number of carbon atoms for all the hydrophobic chains of a single group being greater than or equal to 24.

18. Composition according to claim 17, wherein the micelle comprises phospholipids.

19. Composition according to claim 17, wherein the hydrophilic group is a polysaccharide.

20. Composition according to claim 19, wherein the polysaccharide is chosen from agarose, dextran, starch, cellulose, amylose or amylopectin.

21. Composition according to either claim 17, wherein the hydrophilic group is a copolymer of polyethylene glycol.

22. Composition according to claim 1, characterized in that it is a nail varnish.

23. Composition according to claim 1, characterized in that it is a lacquer.

24. Composition according to claim 1, characterized in that it is a cream.

25. Method for preparing a composition according to claim 1, comprising steps consisting of
i) provision of fluorescent nanoparticles;
ii) if necessary, a previously compatibility treatment of the fluorescent nanoparticles; and
iii) introduction of the fluorescent nanoparticles treated in this way into a cosmetic vehicle.
